SEQUENCE LISTING (19 sequences)

SEQ ID NO: 1 - DNA, 1815 bp, Hendra virus
  CDS (1)..(1815)
atgatggctgattccaaattggtaagcctgaacaataatctatctggtaaaatcaaggatcaaggtaaagttatcaagaattattacggcacaatggacatcaagaaaattaacgatgggttattagatagtaagatacttggggcgtttaacacagtgatagctttgttgggatcaatcatcatcattgtgatgaatatcatgataattcaaaattacaccagaacgactgataatcaggcactaatcaaagagtcactccagagtgtacagcaacaaatcaaagctttaacagacaaaatcgggacagagataggccccaaagtctcactaattgacacatccagcaccatcacaattcctgctaacatagggttactgggatccaagataagtcagtctaccagcagtattaatgagaatgttaacgataaatgcaaatttactcttcctcctttaaagattcatgagtgtaatatctcttgtccgaatcctttgcctttcagagaataccgaccaatctcacaaggggtgagtgatcttgtaggactgccgaaccagatctgtctacagaagacaacatcaacaatcttaaagcccaggctgatatcctatactctaccaattaataccagagaaggggtttgcatcactgacccacttttggctgttgataatggcttcttcgcctatagccatcttgaaaagatcggatcatgtactagaggaattgcaaaacaaaggataataggggtgggtgaggtattggataggggtgataaggtgccatcaatgtttatgaccaatgtttggacaccacccaatccaagcaccatccatcattgcagctcaacttaccatgaagatttttattacacattgtgcgcagtgtcccatgtgggagatcctatccttaacagtacttcctggacagagtcactgtctctgattcgtcttgctgtaagaccaaaaagtgatagtggagactacaatcagaaatacatcgctataactaaagttgaaagagggaagtacgataaggtgatgccttacggtccatcaggtatcaagcaaggggatacattgtactttccggccgtcggttttttgccaaggaccgaatttcaatataatgactctaattgtcccataattcattgcaagtacagcaaagcagaaaactgtaggctttcaatgggtgtcaactccaaaagtcattatattttgagatcaggactattgaagtataatctatctcttggaggagacatcatactccaatttatcgagattgctgacaatagattgaccatcggttctcctagtaagatatacaattccctaggtcaacccgttttctaccaggcatcatattcttgggatacgatgattaaattaggcgatgttgataccgttgaccctctaagagtacagtggagaaataacagtgtgatttctagacctggacagtcacagtgtcctcgatttaatgtctgtcccgaggtatgctgggaagggacatataatgatgcttttctaatagaccggctaaactgggttagtgctggtgtttatttaaacagtaaccaaactgcagagaaccctgtgtttgccgtattcaaggataacgagatcctttaccaagttccactggctgaagatgacacaaatgcacaaaaaaccatcacagattgcttcttgctggagaatgtcatatggtgtatatcactagtagaaatatacgatacaggagacagtgtgataaggccaaaactatttgcagtcaagatacctgcccaatgttcagagagttga

SEQ ID NO: 2 - protein, 604 aa, Hendra virus
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 3 - DNA, 1809 bp, Nipah virus
  CDS (1)..(1809)
atgccggcagaaaacaagaaagttagattcgaaaatactacttcagacaaagggaaaattcctagtaaagttattaagagctactacggaaccatggacattaagaaaataaatgaaggattattggacagcaaaatattaagtgctttcaacacagtaatagcattgcttggatctatcgtgatcatagtgatgaatataatgatcatccaaaattacacaagatcaacagacaatcaggccgtgatcaaagatgcgttgcagggtatccaacagcagatcaaagggcttgctgacaaaatcggcacagagatagggcccaaagtatcactgattgacacatccagtaccattactatcccagctaacattgggctgttaggttcaaagatcagccagtcgactgcaagtataaatgagaatgtgaatgaaaaatgcaaattcacactgcctcccttgaaaatccacgaatgtaacatttcttgtcctaacccactcccttttagagagtataggccacagacagaaggggtgagcaatctagtaggattacctaataatatttgcctgcaaaagacatctaatcagatattgaagccaaagctgatttcatacactttacccgtagtcggtcaaagtggtacctgtatcacagacccattgctggctatggacgagggctattttgcatatagccacctggaaagaatcggatcatgttcaagaggggtctccaaacaaagaataataggagttggagaggtactagacagaggtgatgaagttccttctttatttatgaccaatgtctggaccccaccaaatccaaacaccgtttaccactgtagtgctgtatacaacaatgaattctattatgtactttgtgcagtgtcaactgttggagaccctattctgaatagcacctactggtccggatctctaatgatgacccgtctagctgtgaaacccaagagtaatggtgggggttacaatcaacatcaacttgccctacgaagtatcgagaaagggaggtatgataaagttatgccgtatggaccttcaggcatcaaacagggtgacaccctgtattttcctgctgtaggatttttggtcaggacagagtttaaatacaatgattcaaattgtcccatcacgaagtgtcaatacagtaaacctgaaaattgcaggctatctatggggattagaccaaacagccattatatccttcgatctggactattaaaatacaatctatcagatggggagaaccccaaagttgtattcattgaaatatctgatcaaagattatctattggatctcctagcaaaatctatgattctttgggtcaacctgttttctaccaagcgtcattttcatgggatactatgattaaatttggagatgttctaacagtcaaccctctggttgtcaattggcgtaataacacggtaatatcaagacccgggcaatcacaatgccctagattcaatacatgtccagagatctgctgggaaggagtttataatgatgcattcctaattgacagaatcaattggataagcgcgggtgtattccttgacagcaatcagaccgcagaaaatcctgtttttactgtattcaaagataatgaaatactttatagggcacaactggcttctgaggacaccaatgcacaaaaaacaataactaattgttttctcttgaagaataagatttggtgcatatcattggttgagatatatgacacaggagacaatgtcataagacccaaactattcgcggttaagataccagagcaatgtacataa

SEQ ID NO: 4 - protein, 602 aa, Nipah virus
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 5 - DNA, 40 bp, Artificial Sequence
  Primer
gtcgaccaccatgcaaaattacaccagaacgactgataat

SEQ ID NO: 6 - DNA, 45 bp, Artificial Sequence
  Primer
gtttaaacgtcgaccaatcaactctctgaacattgggcaggtatc

SEQ ID NO: 7 - DNA, 39 bp, Artificial Sequence
  Primer
ctcgagcaccatgcaaaattacacaagatcaacagacaa

SEQ ID NO: 8 - DNA, 45 bp, Artificial Sequence
  Primer
ctcgagtagcagccggatcaagcttatgtacattgctctggtatc

SEQ ID NO: 9 - DNA, 46 bp, Artificial Sequence
  Synthetic oligonucleotide
caaggagaccgctgctgctaagttcgaacgccagcacatggattct

SEQ ID NO: 10 - DNA, 54 bp, Artificial Sequence
  Synthetic oligonucleotide
aattagaatccatgtgctggcgttcgaacttagcagcagcggtctccttggtac

SEQ ID NO: 11 - DNA, 31 bp, Artificial Sequence
  Synthetic oligonucleotide
cgaacaaaagctcatctcagaagaggatctg

SEQ ID NO: 12 - DNA, 39 bp, Artificial Sequence
  Synthetic oligonucleotide
aattcagatcctcttctgagatgagcttttgttcggtac

SEQ ID NO: 13 - DNA, 34 bp, Artificial Sequence
  Synthetic oligonucleotide
tcgacccaccatggagacagacacactcctgcta

SEQ ID NO: 14 - DNA, 1662 bp, Artificial Sequence
  HeV sG viral sequence
  CDS (1)..(1662)
atggaaaccgacaccctgctgctgtgggtgctgctcctgtgggtccccggcagcacaggcgactacaccagaacgactgataatcaggcactaatcaaagagtcactccagagtgtacagcaacaaatcaaagctttaacagacaaaatcgggacagagataggccccaaagtctcactaattgacacatccagcaccatcacaattcctgctaacatagggttactgggatccaagataagtcagtctaccagcagtattaatgagaatgttaacgataaatgcaaatttactcttcctcctttaaagattcatgagtgtaatatctcttgtccgaatcctttgcctttcagagaataccgaccaatctcacaaggggtgagtgatcttgtaggactgccgaaccagatctgtctacagaagacaacatcaacaatcttaaagcccaggctgatatcctatactctaccaattaataccagagaaggggtttgcatcactgacccacttttggctgttgataatggcttcttcgcctatagccatcttgaaaagatcggatcatgtactagaggaattgcaaaacaaaggataataggggtgggtgaggtattggataggggtgataaggtgccatcaatgtttatgaccaatgtttggacaccacccaatccaagcaccatccatcattgcagctcaacttaccatgaagatttttattacacattgtgcgcagtgtcccatgtgggagatcctatccttaacagtacttcctggacagagtcactgtctctgattcgtcttgctgtaagaccaaaaagtgatagtggagactacaatcagaaatacatcgctataactaaagttgaaagagggaagtacgataaggtgatgccttacggtccatcaggtatcaagcaaggggatacattgtactttccggccgtcggttttttgccaaggaccgaatttcaatataatgactctaattgtcccataattcattgcaagtacagcaaagcagaaaactgtaggctttcaatgggtgtcaactccaaaagtcattatattttgagatcaggactattgaagtataatctatctcttggaggagacatcatactccaatttatcgagattgctgacaatagattgaccatcggttctcctagtaagatatacaattccctaggtcaacccgttttctaccaggcatcatattcttgggatacgatgattaaattaggcgatgttgataccgttgaccctctaagagtacagtggagaaataacagtgtgatttctagacctggacagtcacagtgtcctcgatttaatgtctgtcccgaggtatgctgggaagggacatataatgatgcttttctaatagaccggctaaactgggttagtgctggtgtttatttaaacagtaaccaaactgcagagaaccctgtgtttgccgtattcaaggataacgagatcctttaccaagttccactggctgaagatgacacaaatgcacaaaaaaccatcacagattgcttcttgctggagaatgtcatatggtgtatatcactagtagaaatatacgatacaggagacagtgtgataaggccaaaactatttgcagtcaagatacctgcccaatgttcagagagttga

SEQ ID NO: 15 - protein, 553 aa, Artificial Sequence
  Synthetic Construct
METDTLLLWVLLLWVPGSTGDYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 16 - DNA, 1662 bp, Artificial Sequence
  Mammalian - Codon optimized Hendra virus sG
  CDS (1)..(1662)
atggaaaccgacaccctgctgctgtgggtgctgctcctgtgggtccccggcagcacaggcgactacacccggaccaccgacaaccaggccctgatcaaagagtccctgcagagcgtccagcagcagatcaaggccctgaccgacaagatcggcaccgagatcggccccaaagtgtccctgatcgacaccagcagcaccatcaccatccccgccaacatcgggctgctgggctccaagatcagccagagcaccagctccatcaacgagaacgtgaacgacaagtgcaagttcaccctgccccccctgaagatccacgagtgcaacatcagctgccccaaccccctgcccttccgggagtaccggcccatcagccagggcgtgagcgacctggtgggcctgcccaaccagatctgcctgcagaaaaccacctccaccatcctgaagccccggctgatcagctacaccctgcccatcaacacccgggagggcgtgtgcatcaccgaccctctgctggccgtggacaacggcttcttcgcctacagccacctggaaaagatcggcagctgcacccggggcattgccaagcagcggatcatcggcgtgggcgaggtgctggaccggggcgacaaggtgcccagcatgttcatgaccaacgtgtggaccccccccaaccccagcacaatccaccactgcagcagcacctaccacgaggacttctactacaccctgtgcgccgtgagccacgtgggcgaccccatcctgaacagcaccagctggaccgagagcctgagcctgatccggctggccgtgcggcccaagagcgacagcggcgactacaaccagaagtatatcgccatcaccaaggtggagcggggcaagtacgacaaagtgatgccctacggccccagcggcatcaagcagggcgacacactgtacttccccgccgtgggcttcctgccccggaccgagttccagtacaacgacagcaactgccccatcatccactgcaagtacagcaaggccgagaactgcagactgagcatgggcgtgaacagcaagagccactacatcctgcggagcggcctgctgaagtacaacctgtccctgggcggcgacatcatcctgcagttcatcgagatcgccgacaaccggctgaccatcggcagccccagcaagatctacaacagcctgggccagcccgtgttctaccaggccagctacagctgggacaccatgatcaagctgggggacgtggacaccgtggaccccctgcgggtgcagtggcggaacaacagcgtgatcagcagacccggccagagccagtgcccccggttcaacgtgtgccccgaagtgtgctgggagggcacctacaacgacgcctttctgatcgaccggctgaactgggtgtccgccggagtgtacctgaactccaaccagaccgccgagaaccccgtgttcgccgtgttcaaggacaacgagatcctgtaccaggtgcccctggccgaggacgacaccaacgcccagaaaaccatcaccgactgctttctgctggaaaacgtgatctggtgcatcagcctggtggagatctacgacaccggcgactccgtgatccggcccaagctgtttgccgtgaagatccccgcccagtgcagcgagagctga

SEQ ID NO: 17 - protein, 553 aa, Artificial Sequence; identical to SEQ ID NO: 15
  Synthetic Construct

SEQ ID NO: 18 - DNA, 30 bp, Artificial Sequence
  Artificial primers
gatatcgccaccatggaaaccgacaccctg

SEQ ID NO: 19 - DNA, 24 bp, Artificial Sequence
  Artificial primers
ggtacctcagctctcgctgcactg

(19) United States
(12) Patent Application Publication
(10) Pub. No.: US 2019/0083604 A1
(43) Pub. Date: Mar. 21, 2019

(54) HENDRA AND NIPAH VIRUS G GLYCOPROTEIN IMMUNOGENIC COMPOSITIONS

(71) Applicant: Zoetis Services LLC, Parsippany, NJ (US)

(72) Inventors: John M. Hardham, Kalamazoo, MI (US); Jin-An Huang, Parkville (AU); Paul J. Dominowski, Kalamazoo, MI (US)

(21) Appl. No.: 16/014,029

(22) Filed: Jun. 21, 2018

Related U.S. Application Data
(63) Continuation of application No. 14/915,813, filed on Mar. 1, 2016, now abandoned.
(60) Provisional application No. 61/874,085, filed on Sep. 5, 2013.

(51) Int. Cl. A61K 39/155; A61K 39/12; G01N 33/569; C12N 7/00; A61K 39/00

(52) U.S. Cl. CPC A61K 39/155; A61K 2039/545; G01N 33/56983; C12N 7/00; A61K 2039/55561; C12N 2760/18234; A61K 2039/58; A61K 2039/552; A61K 2039/543; A61K 2039/54; C12N 2760/18222; C12N 2760/18233; G01N 2333/115; A61K 2039/55505; A61K 2039/55566; G01N 2469/20; A61K 39/12

(57) ABSTRACT

This invention relates to Hendra virus and Nipah virus immunogenic compositions and methods of use. The invention further relates to immunogenic compositions comprising Hendra virus G glycoprotein, and methods of protecting against Nipah virus infection and disease. The invention also relates to methods of distinguishing subjects vaccinated with the immunogenic compositions of the invention from those infected with Hendra and/or Nipah virus.

### FIELD OF THE INVENTION

The present invention relates to immunogenic and vaccine compositions comprising a G glycoprotein from Hendra virus (HeV) and/or Nipah virus (NiV) and to methods of use relating thereto. The present invention also relates to compositions comprising a G glycoprotein from HeV useful in protecting against infection and disease cause by NiV.

### BACKGROUND OF THE INVENTION

Recurrent outbreaks of NiV resulting in significant numbers of human fatalities have recently been problematic (see e.g. Butler (2000) Nature 429, 7; Gurley et al. (2007) Emerging Infectious Diseases 13(7), 1031-1037). Case studies have linked disease in humans to zoonotic transmission from swine (see e.g. Parashar et al. (2000) J Infect Dis. 181, 1755-1759). HeV is also known to cause fatalities in human and animals and is genetically and immunologically closely related to NiV. Both Nipah virus and Hendra virus are United States, National Institute of Allergy and Infectious Disease, category C priority agents of biodefense concern, and are USDA-APHIS High-Consequence Tier 3 Foreign Animal Disease agents, meaning they are given high consideration in program priorities with respect to countermeasure stockpile requirements.

There is presently one licensed vaccine for the prevention of infection or disease caused by Hendra virus (Equivac® HeV; Zoetis); no licensed vaccine exists for preventing Nipah virus infection, however. As these viruses are zoonotic Biological Safety Level-4 agents (BSL-4), production of vaccines and/or diagnostics, coupled with safety concerns, is very costly and difficult.

Various groups have demonstrated that recombinant G protein vaccines based on antigen derived from either HeV (see e.g. McEachern et al. (2008) Vaccine 26, 3842-3852; Bossart et al. (2012) Sci Transl Med. 4(146), 1-8), or from NiV (see e.g. Mungall et al. (2006) J Virol. 80(24), 12293-12302; Weingartl et al. (2006) J Virol. 80(16), 7929-7938) can provide protection against infection. However, none have yet been successful in commercializing any of these vaccines. Thus, there remains a need for Nipah virus or Hendra virus vaccines and diagnostics that allow for high throughput production.

Paramyxoviruses such as HeV and NiV possess two major membrane-anchored glycoproteins in the envelope of the viral particle. One glycoprotein is required for virion attachment to receptors on host cells and is designated as either hemagglutinin-neuraminidase protein (HN) or hemagglutinin protein (H), and the other is glycoprotein (G), which has neither hemagglutination nor neuraminidase activities. The attachment glycoproteins are type II membrane proteins, where the molecule's amino (N) terminus is oriented toward the cytoplasm and the protein's carboxy (C) terminus is extracellular. The other major glycoprotein is the fusion (F) glycoprotein, which is a trimeric class I fusogenic envelope glycoprotein containing two heptad repeat (HR) regions and a hydrophobic fusion peptide. HeV and NiV infect cells though a pH-independent membrane fusion process into receptive host cells through the concerted action of their attachment G glycoprotein and F glycoprotein following receptor binding. The primary function of the HeV and NiV attachment G glycoprotein is to engage appropriate receptors on the surfaces of host cells, which for the majority of well-characterized paramyxoviruses are sialic acid moieties. The HeV and NiV G glycoproteins utilize the host cell protein receptors ephrin B2 and/or ephrin B3, and antibodies have been developed which block viral attachment by the G glycoprotein (WO2006137931, Bishop (2008) J. Virol. 82: 11398-11409). Further, vaccines have been developed which also use the G glycoprotein as a means for generating an immunoprotective response against HeV and NiV infection (WO2009117035). That Hendra virus G glycoprotein can potentially cross protect against infection by Nipah virus is reasonably suggested by K. Bossart et al. (Journal of Virology, vol 79, pp 6690-6702, 2005) and B. Mungall et al. (Journal of Virology, vol 80, pp. 12293-12302, 2006). Of course, the problem that remains to be solved is the provision of highly effective vaccine compositions that enhance cross protection and make it both highly effective, and medically and commercially practical.

The combination of HeV and/or NiV G glycoproteins with a biologically-acceptable adjuvant in a vaccine represents an advancement in developing effective HeV and NiV vaccines, given the potential for enhanced immunoreactivity with decreased adjuvant side effects when these components are administered in combination.

### SUMMARY OF THE INVENTION

The invention encompasses an immunogenic composition comprising Hendra and/or Nipah virus G protein, an adjuvant, and one or more excipients, in an amount effective to elicit production of neutralizing antibodies against the Hendra and/or Nipah virus following administration to a subject.

In some embodiments, soluble Hendra virus G glycoprotein consists of amino acids 73 to 604 of the native Hendra G glycoprotein (SEQ ID NO: 2). In some embodiments, the soluble Hendra virus G glycoprotein is encoded by a nucleotide sequence comprising nucleotides 64 to 1662 of SEQ ID NO: 16. In some embodiments, the soluble Hendra virus G protein is present in dimer form wherein each soluble Hendra virus G glycoprotein dimer subunit is connected by one or more disulfide bonds. In some embodiments, the soluble Hendra virus G protein is present in tetramer form. In some embodiments, the tetramer form exists as a dimer of dimers non-covalently linked and/or connected by one or more disulfide bonds. In some embodiments, the concentration of soluble Hendra virus G protein can be about 5 to 250 μg/ml in the immunogenic composition (see also WO2006/085979)

In some embodiments, the adjuvant may be an oil-in-water emulsion.

In some embodiments, the adjuvant may be SP-Oil.

In some embodiments, the adjuvant may comprise a saponin, a sterol, a quaternary ammonium compound, a polymer, a glycolipid, and an immunostimulatory oligonucleotide.

In some embodiments, the invention also encompasses a method of producing a neutralizing antibody response against a Hendra and/or Nipah virus in a subject comprising administering to the subject the immunogenic composition described herein in an amount and duration effective to produce the neutralizing antibody response. In some embodiments, the neutralizing antibody response reduces Hendra and/or Nipah virus reproduction in the subject and may also reduce Hendra and/or Nipah virus shedding in the subject. In some embodiments, the subject has been exposed to Hendra and/or Nipah virus while in other embodiments, the subject is suffering from a Hendra and/or Nipah virus infection. In some embodiments, the invention encompasses a method of producing a neutralizing antibody response against a Hendra virus in a subject comprising administering to the subject the immunogenic composition described herein in an amount and duration effective to produce the neutralizing antibody response. In some embodiments, the invention encompasses a method of producing a neutralizing antibody response against a Nipah virus in a subject comprising administering to the subject the immunogenic composition described herein in an amount and duration effective to produce the neutralizing antibody response.

In some embodiments, the immunogenic composition is administered intramuscularly. In some embodiments, the immunogenic composition is administered in multiple doses and the first dose is followed by a second dose at least about twenty-one days to about twenty-eight days after the first dose. In some embodiments, each dose contains about 50, about 100, or about 250 μg of soluble Hendra virus G protein.

The invention further encompasses a method of differentiating a subject vaccinated with the immunogenic composition described herein from a subject exposed to Hendra and/or Nipah virus comprising detecting the presence of an antibody in a biological sample isolated from the subject against at least one of any of the following HeV and/or NiV viral proteins selected from the group consisting of fusion protein (F), matrix protein (M), phosphoprotein (P), large protein (L) and nucleocapsid protein (N).

The immunogenic compositions and methods of the invention can be administered to a subject such as a human, horse, cow, sheep, pig, goat, chicken, dog or cat.

The invention also encompasses a method of producing a neutralizing antibody response against a Hendra and/or Nipah virus in a human subject comprising administering to the subject an immunogenic composition comprising a Hendra virus soluble G glycoprotein in an amount and duration effective to produce the neutralizing antibody response. In some embodiments, the immunogenic composition further comprises an adjuvant. In regard of Hendra virus G glycoprotein polypeptides useful in the practice of the invention, recombinant expression thereof, and formulation into vaccines compositions, the entire disclosure of published international patent applications WO 2012/158643 and WO2006/085979 is incorporated by reference herein, as if fully set forth.

### BRIEF DESCRIPTION OF THE FIGURES

FIG. 1 depicts a schematic diagram of sGHeV vaccination and NiV challenge schedule. Dates of sGHeV vaccination, NiV challenge and euthanasia are indicated by arrows. Blood and swab specimens were collected on days −42, −7, 0, 3, 5, 7, 10, 14, 21 and 28 post-challenge as indicated (*). Gray text denotes challenge timeline (top row); black text denotes vaccination timeline (bottom row). African green monkey (AGM) number for subjects in each vaccine dose group and one control subject are shown.

FIG. 2 depicts the survival curve of NiV-infected subjects. Data from control subjects (n=2) and sGHeV vaccinated subjects (n=9) were used to generate the Kaplan-Meier survival curve. Control included data from one additional historical control subject. Vaccinated subjects received 10 μg, 50 μg or 100 μg sGHeV administered subcutaneously twice. Average time to end stage disease was 11 days in control subjects whereas all vaccinated subjects survived until euthanasia at the end of the study.

FIG. 3 depicts NiV- and HeV-specific Immunoglobulin (Ig) in vaccinated subjects. Serum and nasal swabs were collected from vaccinated subjects and IgG, IgA and IgM responses were evaluated using sGHeV, and sGNiV multiplexed microsphere assays. Sera or swabs from subjects in the same vaccine dose group (n=3) were assayed individually and the mean of microsphere median fluorescence intensities (M.F.I.) was calculated which is shown on the Y-axis. Error bars represent the standard error of the mean. Serum sG-specific Ig is shown in black (sGHeV (open triangles), sGNiV (solid triangles)) and mucosal sG-specific IgA is shown in gray symbols (sGHeV (open triangles), sGNiV (solid triangles)).

### BRIEF DESCRIPTION OF THE SEQUENCE LISTING

SEQ ID NO: 1 provides the amino acid sequence of native Hendra G glycoprotein, and corresponding codons.

SEQ ID NO: 2 provides the amino acid sequence of native Hendra G glycoprotein.

SEQ ID NO: 3 provides the amino acid sequence of native Nipah G glycoprotein, and corresponding codons.

SEQ ID NO: 4 provides the amino acid sequence encoding Nipah G glycoprotein.

SEQ ID NO: 5 provides an artificial primer, see Example 1.

SEQ ID NO: 6 provides an artificial primer, see Example 1.

SEQ ID NO: 7 provides an artificial primer, see Example 1.

SEQ ID NO: 8 provides an artificial primer, see Example 1.

SEQ ID NO: 9 provides an artificial primer, see Example 1.

SEQ ID NO: 10 provides an artificial primer, see Example 1.

SEQ ID NO: 11 provides an artificial primer, see Example 1.

SEQ ID NO: 12 provides an artificial primer, see Example 1.

SEQ ID NO: 13 provides an artificial primer, see Example 1.

SEQ ID NO: 14 provides an additional Hendra G protein soluble fragment amino acid sequence, and corresponding nucleotide sequence.

SEQ ID NO: 15 provides Hendra G glycoprotein with an Ig(kappa) leader sequence.

SEQ ID NO: 16 provides a codon-optimized nucleotide sequence encoding soluble Hendra G glycoprotein.

SEQ ID NO: 17 provides an additional Hendra G protein sequence.

SEQ ID NO: 18 provides an artificial primer, see Example 1.

SEQ ID NO: 19 provides an artificial primer, see Example 1.

### DETAILED DESCRIPTION OF THE INVENTION

### Vaccine & Immunogenic Compositions

The vaccine and immunogenic composition of the present invention induces at least one of a number of humoral and cellular immune responses in a subject who has been administered the composition or is effective in enhancing at least one immune response against at least one strain of HeV and/or NiV, such that the administration is suitable for vaccination purposes and/or prevention of HeV and/or NiV infection by one or more strains of HeV and/or NiV. The composition of the present invention delivers to a subject in need thereof a G glycoprotein, including soluble G glycoproteins from HeV and/or NiV and an adjuvant. In some embodiments, the amount of G glycoprotein includes, but is not limited to, 5, 10, 15, 20, 25, 30, 35, 40, 45, 50, 75, 100, 150, 200 or 250 μg per ml. For swine, the recommended amount of G glycoprotein antigen is between about 5 and 100 μg per dose, each dose being preferably from about 0.5 to about 2.0 ML. Preferably, 2 doses are given, for example, between 2 weeks and 3 months apart, with the duration of protective immunity extending out to one year, or longer. According to the practice of the invention, piglets may also be vaccinated, pre- or post-weaning (i.e. before or after about 21 days of life).

A. HeV and NiV G Proteins. In some embodiments, the vaccine and immunogenic compositions comprise one or more HeV and/or NiV G glycoproteins as described herein. The term protein is used broadly herein to include polypeptide or fragments thereof. By way of example, and not limitation, a HeV G glycoprotein may in soluble form and comprise amino acids 73-604 of the amino acid sequence for a HeV G glycoprotein in Wang (2000) J. Virol. 74, 9972-9979 (see also Yu (1998) Virology 251, 227-233). Also by way of example and not limitation, a NiV G glycoprotein may be in soluble form and comprise amino acids 71-602 of the amino acid sequence for a NiV G glycoprotein in Harcourt (2000) Virology 271: 334-349, 2000 (see also Chua (2000) Science, 288, 1432-1).

Generally, the soluble forms of the HeV and NiV G glycoproteins comprise all or part of the ectodomain (e.g. extracellular) of the G glycoprotein of a HeV or NiV and are generally produced by deleting all or part of the transmembrane domain of the G glycoprotein and all or part of the cytoplasmic tail of the G glycoprotein. By way of example, a soluble G glycoprotein may comprise the complete ectodomain of a HeV or NiV G glycoprotein. Also by way of example, and not limitation a soluble G glycoprotein may comprise all or part of the ectodomain and part of the transmembrane domain of a HeV or NiV G glycoprotein.

The soluble HeV or NiV G glycoproteins of the invention, generally retain one or more characteristics of the corresponding native viral glycoprotein, such as, ability to interact or bind the viral host cell receptor, can be produced in oligomeric form or forms, or the ability to elicit antibodies (including, but not limited to, viral neutralizing antibodies) capable of recognizing native G glycoprotein. Examples of additional characteristics include, but are not limited to, the ability to block or prevent infection of a host cell. Conventional methodology may be utilized to evaluate soluble HeV or NiV G glycoproteins for one of more of the characteristics.

By way of example, and not limitation, a polynucleotide encoding a soluble HeV G glycoprotein may comprise a polynucleotide sequence encoding about amino acids 73-604 of the amino acid sequence for an HeV G glycoprotein in Wang (2000) J. Virol. 74, 9972-9979 (SEQ ID NO: 2). Also by way of example, and not limitation, a polynucleotide encoding a soluble HeV G glycoprotein may comprise nucleotides 9129 to 10727 of the polynucleotide sequence for an HeV G glycoprotein in Wang (2000) J. Virol. 74, 9972-9979. In addition, codon optimized polynucleotide sequence encoding about amino acids 73-604 of the amino acid sequence for an HeV G glycoprotein (SEQ ID NO: 2) can also be utilized. In some embodiments, these codon optimized sequences comprises or consist of nucleotides 64 to 1662 of SEQ ID NO: 16. In further embodiments, the codon optimized sequences comprises or consists of SEQ ID NO: 16 which includes nucleotides encoding an lgk leader sequence.

By way of example, and not limitation, a NiV G glycoprotein may in soluble form and comprise amino acids 71-602 of the amino acid sequence for the NiV G glycoprotein in Harcourt (2000) Virology 271, 334-349. Non-limiting examples of sequences that may be used to construct a soluble NiV G glycoprotein can be found in Harcourt (2000) Virology 271, 334-349. Generally, G glycoprotein sequences from any Nipah virus isolate or strain may be utilized to derive the polynucleotides and polypeptides of the invention.

By way of example, and not limitation, a polynucleotide encoding a soluble NiV G glycoprotein may comprise a polynucleotide sequence encoding about amino acids 71-602 of the amino acid sequence for an NiV G Glycoprotein in Harcourt (2000) Virology 271, 334-349. Also by way of example, and not limitation, a polynucleotide encoding a soluble NiV G glycoprotein may comprise 234-2042 of the polynucleotide sequence for an NiV G glycoprotein in Harcourt (2000) Virology 271, 334-349 (SEQ ID NO: 4). In addition, codon optimized polynucleotide sequence encoding about amino acids 71-602 of the amino acid sequence for an NiV G glycoprotein can also be utilized.

Functional equivalents of these G glycoproteins can be used in the immunogenic and vaccine compositions of the invention. By way of example and not limitation functionally equivalent polypeptides possess one or more of the following characteristics: ability to interact or bind the viral host cell receptor, can be produced in dimeric or tetrameric form or forms, the ability to elicit antibodies (including, but not limited to, HeV and/or NiV viral neutralizing antibodies) capable of recognizing native G glycoprotein and/or the ability to block or prevent infection of a host cell.

In some embodiments, the G glycoprotein may be in dimeric and/or tetrameric form. Such dimers depend upon the formation of disulfide bonds formed between cysteine residues in the G glycoprotein. Such disulfide bonds can correspond to those formed in the native G glycoprotein (e.g. location of cyteines remains unchanged) when expressed in the surface of HeV or NiV or may be altered in the presence or location (e.g. by altering the location of cysteine(s) in the amino acid sequence) of the G glycoprotein so as to form different dimeric and/or tetrameric forms of the G glycoprotein which enhance antigenicity. Additionally, non-dimerized and tetramerized forms are also within the invention, again taking into account that G glycoprotein presents numerous conformation-dependent epitopes (i.e. that arise from a tertiary three dimensional structure) and that preservation numerous of such natural epitopes is highly preferred so as to impart a neutralizing antibody response.

The HeV immunogenic and vaccine compositions of the invention may contain proteins of variable length but include the amino acid residues 73 to 604 of SEQ ID NO: 2. In one embodiment of the present invention, envelope proteins of the invention are at least about 85, 90, 91, 92, 93, 94, 95, 96, 97, 98, or 99% identical to the HeV glycoprotein of SEQ ID NO: 2 (including amino acids 73 to 604). Accordingly, the HeV G glycoproteins of the invention comprise immunogenic fragments of the native HeV G glycoprotein with sufficient number of amino acids to produce conformational epitopes. Non-limiting examples of immunogenic fragments include amino acid sequences which may be at least 530, 531, 532, 533, 534 or 535 or more amino acids in length. In some embodiments, the HeV G glycoprotein comprises or consists of SEQ ID NO: 2 or synthetic constructs further comprising an IgK leader sequence (SEQ ID NO: 15).

The NiV immunogenic and vaccine compositions of the invention may contain proteins of variable length but include the amino acid residues 71 to 602 of SEQ ID NO: 4. In one embodiment of the present invention, envelope proteins of the invention are at least about 85, 90, 91, 92, 93, 94, 95, 96, 97, 98, or 99% identical to the NiV glycoprotein of SEQ ID NO: 4 (including amino acids 71 to 602). Accordingly, the NiV G glycoproteins of the invention comprise immunogenic fragments of the native NiV G glycoprotein with sufficient number of amino acids to produce conformational epitopes. Non-limiting examples of immunogenic fragments include amino acid sequences which may be at least 528, 529, 530, 531, 532, or 533 or more amino acids in length. In some embodiments, the NiV G glycoprotein comprises or consists of SEQ ID NO: 4 or synthetic constructs further comprising a leader sequence.

Immunogenic fragments as described herein will contain at least one epitope of the antigen and display HeV and/or NiV antigenicity and are capable of raising an immune response when presented in a suitable construct, such as for example when fused to other HeV and/or NiV antigens or presented on a carrier, the immune response being directed against the native antigen. In one embodiment of the present invention, the immunogenic fragments contain at least 20 contiguous amino acids from the HeV and/or NiV antigen, for example, at least 50, 75, or 100 contiguous amino acids from the HeV and/or NiV antigen.

HeV and NiV G glycoprotein embodiments further include an isolated polypeptide comprising an amino acid sequence having at least a 85, 90, 91, 92, 93, 94, 95, 96, 97, 98, 99 or 100% identity to native HeV or NiV G glycoproteins, wherein said polypeptide sequence may be identical to the native HeV or NiV G glycoprotein amino acid sequence or may include up to a certain integer number of amino acid alterations as compared to the native HeV or NiV G protein amino acid sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the native HeV or NiV G glycoprotein amino acid sequence.

Sequence identity or homology at the amino acid sequence level can be determined by BLAST (Basic Local Alignment Search Tool) analysis using the algorithm employed by the programs blastp, blastn, blastx, tblastn and tblastx (Altschul (1997) Nucleic Acids Res. 25, 3389-3402 and Karlin (1990) Proc. Natl. Acad. Sci. USA 87, 2264-2268) which are tailored for sequence similarity searching. The approach used by the BLAST program is to first consider similar segments, with gaps (non-contiguous) and without gaps (contiguous), between a query sequence and a database sequence, then to evaluate the statistical significance of all matches that are identified and finally to summarize only those matches which satisfy a preselected threshold of significance. For a discussion of basic issues in similarity searching of sequence databases, see Altschul (1994) Nature Genetics 6, 119-129. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff (1992) Proc. Natl. Acad. Sci. USA 89, 10915-10919), recommended for query sequences over 85 amino acids in length.

The vaccine and immunogenic compositions of the present invention may further comprise additional HeV and/or NiV G proteins from different strains that may further potentiate the immunization methods of the invention.

B. Adjuvants

Generally this invention provides immunogenic compositions, including vaccine compositions, comprising soluble forms of HeV and/or NiV G glycoprotein envelope protein in combination with an adjuvant, and methods for using these compositions for preventing and treating HeV and/or NiV infections in a subject. In the present invention, the vaccine and/or immunogenic composition comprise an adjuvant. As used herein, “adjuvant” refers to an agent which, while not having any specific antigenic effect in itself, may stimulate the immune system, increasing the response to an antigen.

The concentration of adjuvant employed in the compositions described herein will depend upon the nature of the adjuvant. Adjuvants are typically present in the compositions described herein at a final concentration of about 1-50% (v/v) and more typically at a final concentration of about 10%, 15%, 20%, 25%, or 30% (v/v). In compositions comprising SP-Oil, the adjuvant is typically present at between about 1% and about 25% (v/v), more typically between about 5% and about 15% (v/v) such as, for example, at about 10% (v/v). In compositions comprising an acrylic acid polymer and a mixture of a metabolizable oil that comprises one or more terpene hydrocarbon(s) and a polyoxyethylene-polypropylene block copolymer, the ratio of acrylic acid polymer to metabolizable oil/polyoxyethylene-polypropylene block copolymer mixture is typically in a ratio of between about 1:25 and about 1:50 and typically at a final concentration of between about 1% and about 25% (v/v).

In one embodiment, the biologically acceptable adjuvant comprises SP-Oil. SP-Oil is a fluidized oil emulsion which includes a polyoxyethylene-polyoxypropylene block copolymer (Pluronic® L121, BASF Corporation), squalane, polyoxyethylene sorbitan monooleate (Tween® 80, ICI Americas), and a buffered salt solution. SP-Oil is an effective vaccine adjuvant, and is able to induce both a cell-mediated (CMI) and humoral immune response when administered to a subject (see e.g. U.S. Pat. No. 5,709,860).

Polyoxyethylene-polyoxypropylene block copolymers are surfactants that aid in suspending solid and liquid components. These surfactants are commercially available as polymers under the trade name Pluronic®. The preferred surfactant is poloxamer 401 which is commercially available under the trade name Pluronic® L121. In general, the SP-Oil emulsion is an immunostimulating adjuvant mixture which will comprise about 1 to 3% vol/vol of block copolymer, about 2 to 6% vol/vol of squalane, more particularly about 3 to 6% of squalane, and about 0.1 to 0.5% vol/vol of polyoxyethylene sorbitan monooleate, with the remainder being a buffered salt solution.

In one embodiment, the SP-Oil is present at a concentration of between about 1% and about 25% v/v. In one embodiment, the SP-Oil is present at a concentration of between about 5% and about 15% v/v. In one embodiment, the SP-Oil is present at a concentration of about 10% v/v.

In some embodiments the adjuvant may comprise a saponin such as Quil A, a sterol such as cholesterol, a quaternary ammonium compound such as dimethyl dioctadecyl ammonium bromide (DDA), a polymer such as polyacrylic acid (Carbopol®, Lubrizol Corporation), a glycolipid such as N-(2-Deoxy-2-L-leucylamino-b-D-glucopyranosyl)-N-octadecyldodecanoylamide hydroacetate, and an immunostimulatory oligonucleotide, including DNA-based and RNA-based oligonucleotides.

In some embodiments, the saponin for use in the present invention is Quil A and/or its derivatives. Quil A is a saponin preparation isolated from the South American tree Quillaja saponaria Molina, and was first described as having adjuvant activity by Dalsgaard (1974), Saponin adjuvants, Archiv. fur die gesamte Virusforschung, Vol. 44, Springer Verlag, pp. 243-254. Purified fragments of Quil A have been isolated by HPLC which retain adjuvant activity without the toxicity associated with Quil A (EP 0362278), for example QS7 and QS21 (also known as QA7 and QA21). QS21 is a natural saponin derived from the bark of Quillaja saponaria Molina which induces CD8+cytotoxic T cells (CTL), Th1 cells and a predominant IgG2a antibody response, and is a saponin for use in the context of the present invention. Other suitable saponins for use in the adjuvant include, but are not limited to, the QH-A, QH-B and QH-C subfractions of Quil A, those from species other than Quillaja saponaria, such as those from the genera Panax (ginseng), Astragalus, Achyranthes, Soy bean, Acacia and Codonopsis. In some embodiments, the saponin is isolated from a species other than Quillaja saponaria.

In some embodiments the adjuvant may comprise a sterol. Sterols share a common chemical core, which is a steroid ring structure[s] having a hydroxyl (OH) group usually attached to carbon-3. The hydrocarbon chain of the fatty-acid substituent varies in length, usually from 16 to 20 carbon atoms, and can be saturated or unsaturated. Sterols commonly contain one or more double bonds in the ring structure, and also a variety of substituents attached to the rings. Sterols and their fatty-acid esters are essentially water-insoluble. In view of these chemical similarities, it is thus likely that the sterols sharing this chemical core would have similar properties when used in the vaccine compositions of the instant invention. Sterols suitable for use in the adjuvant include cholesterol, β-sitosterol, stigmasterol, ergosterol, and ergocalciferol. These sterols are well known in the art, and can be purchased commercially. For example cholesterol is disclosed in the Merck Index, 12th Ed., p. 369. The amount of sterols suitable for use in the adjuvant depends upon the nature of the sterol used. However, they are generally used in an amount of about 1 μg to about 5,000 μg per dose. They also are used in an amount of about 1 μg to about 4,000 μg per dose, about 1 μg to about 3,000 μg per dose, about 1 μg to about 2,000 μg per dose, and about 1 μg to about 1,000 μg per dose. They are also used in an amount of about 5 μg to about 750 μg per dose, about 5 μg to about 500 μg per dose, about 5 μg to about 200 μg per dose, about 5 μg to about 100 μg per dose, about 15 μg to about 100 μg per dose, and about 30 μg to about 75 μg per dose.

In some embodiments the adjuvant may comprise a quaternary amine compound. These compounds are ammonium-based, with four hydrocarbon groups. In practice, the hydrocarbon groups are generally limited to alkyl or aryl groups. In an embodiment, the quaternary amine compound is composed of four alkyl chains, two of which are C10-C20 alkyls, and the remaining two are C1-C4 alkyls. In one embodiment, the quaternary amine is dimethyldioctadecylammonium bromide (DDA), chloride or pharmaceutically acceptable counterion.

In some embodiments the adjuvant may comprise one or more immunomodulatory agents, such as interleukins, interferons, or other cytokines. These materials can be purchased commercially. The amount of an immunomodulator suitable for use in the adjuvant depends upon the nature of the immunomodulator used and the subject. However, they are generally used in an amount of about 1 μg to about 5,000 μg per dose. They also are used in an amount of about 1 μg to about 4,000 μg per dose, about 1 μg to about 3,000 μg per dose, about 1 μg to about 2,000 μg per dose, and about 1 μg to about 1,000 μg per dose. They are also used in an amount of about 5 μg to about 750 μg per dose, about 5 μg to about 500 μg per dose, about 5 μg to about 200 μg per dose, about 5 μg to about 100 μg per dose, about 15 μg to about 100 μg per dose, and in an amount of about 30 μg to about 75 μg per dose.

In some embodiments the adjuvant may comprise one or more polymers such as, for example, DEAE Dextran, polyethylene glycol, and polyacrylic acid and polymethacrylic acid (eg, CARBOPOL®). Such material can be purchased commercially. The amount of polymers suitable for use in the adjuvant depends upon the nature of the polymers used. However, they are generally used in an amount of about 0.0001% volume to volume (v/v) to about 75% v/v. In other embodiments, they are used in an amount of about 0.001% v/v to about 50% v/v, of about 0.005% v/v to about 25% v/v, of about 0.01% v/v to about 10% v/v, of about 0.05% v/v to about 2% v/v, and of about 0.1% v/v to about 0.75% v/v. In another embodiment, they are used in an amount of about 0.02% v/v to about 0.4% v/v. DEAE-dextran can have a molecular size in the range of 50,000 Da to 5,000,000 Da, or it can be in the range of 500,000 Da to 2,000,000 Da. Such material may be purchased commercially or prepared from dextran.

In some embodiments the adjuvant may comprise a glycolipid. Suitable glycolipids are generally those which activate a Th2 response. The glycolipids include, without limitations, those encompassed by Formula I, and that are generally described in US Publication 20070196384 (Ramasamy et al).

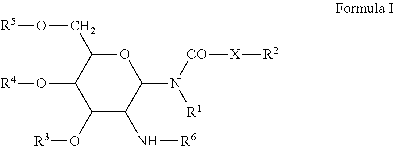

In the structure of Formula I, R1 and R2 are independently hydrogen, or a saturated alkyl radical having up to 20 carbon atoms; X is —CH2-, —O— or —NH—; R2 is hydrogen, or a saturated or unsaturated alkyl radical having up to 20 carbon atoms; R3, R4, and R5 are independently hydrogen,—SO42-, —PO42-, —COC1-10 alkyl; R6 is L-alanyl, L-alpha-aminobutyl, L-arginyl, L-asparginyl, L-aspartyl, L-cysteinyl, L-glutamyl, L-glycyl, L-histidyl, L-hydroxyprolyl, L-isoleucyl, L-leucyl, L-lysyl, L-methionyl, L-ornithinyl, L-phenyalany, L-prolyl, L-seryl, L-threonyl, L-tyrosyl, L-tryptophanyl, and L-valyl or their D-isomers.

In one embodiment, the suitable glycolipid is N-(2-Deoxy-2-L-leucylamino-b-D-glucopyranosyl)-N-octadecyldodecanoylamide or an acetate thereof, also known by the trade name Bay R1005®.

In some embodiments the adjuvant may comprise an immunostimulatory oligonucleotide. Suitable immunostimulatory oligonucleotides include ODN (DNA-based) and ORN (RNA-based) oligonucleotides, which may have modified backbone including, without limitations, phosphorothioate modifications, halogenations, alkylation (e.g., ethyl- or methyl-modifications), and phosphodiester modifications. In some embodiments, poly inosinic-cytidylic acid or derivative thereof (poly I:C) may be used. In a set of embodiments, the oligonucleotides of the instant invention contain palindromes, and preferably, are capable of forming hairpin-like secondary structures comprising a stem and a loop. In certain embodiments, the immunostimulatory oligonucleotides are single-stranded, though they may contain palindromic structures and thus form double-stranded, e.g., stem-loop, structures. Several classes of immunostimulatory oligonucleotides are known in the art.

The amount of immunostimulatory oligonucleotide for use in the adjuvant depends upon the nature of the immunostimulatory oligonucleotide used, and the intended species. However, they are generally used in an amount of about 1 μg to about 20 mg per dose. They also are used in an amount of about 1 μg to about 10 mg per dose, about 1 μg to about 5 mg per dose, about 1 μg to about 4 mg per dose, about μg to about 3 mg per dose, about 1 μg to about 2 mg per dose, and about 1 μg to about 1 mg per dose. They are also used in an amount of about 5 μg to about 750 μg per dose, about 5 μg to about 500 μg per dose, about 5 μg to about 200 μg per dose, about 5 μg to about 100 μg per dose, 10 μg to about 100 μg per dose, about 15 μμg to about 100 μg per dose, and in an amount of about 30 μg to about 75 μg per dose.

In some embodiments the adjuvant may comprise an aluminum-based component. Aluminum is a known adjuvant or a component of adjuvant formulations, and is commercially available in such forms as alhydrogel (Brenntag; Denmark) or REHYDRAGEL® (Reheis, Inc; New Jersey). REHYDRAGEL® is a crystalline aluminum oxyhydroxide, known mineralogically as boehmite. It is effective in vaccines when there is a need to bind negatively-charged proteins. The content of Al₂O₃ ranges from 2% to 10% depending on grade, and its viscosity is 1000-1300 cP. Generally, it may be described as an adsorbent aluminum hydroxide gel.

In some embodiments the present invention includes, but is not limited to, an immunogenic composition comprising an isolated HeV or NiV G protein capable of inducing the production of a cross-reactive neutralizing anti-serum against multiple strains of HeV and/or NiV in vitro, and an adjuvant comprising polyoxyethylene-polyoxypropylene block copolymer (Pluronic® L121), squalane, polyoxyethylene sorbitan monooleate (Tween® 80), and a buffered salt solution, for example wherein the composition contains: 5, 50, 100, or 250 μg of soluble HeV or NiV G protein, and appropriate amounts of the adjuvant components.

In another embodiment of the invention, the vaccine and immunogenic compositions may be part of a pharmaceutical composition. The pharmaceutical compositions of the present invention may contain suitable pharmaceutically acceptable carriers comprising excipients and auxiliaries that facilitate processing of the active compounds into preparations that can be used pharmaceutically for delivery to the site of action.

### C. Excipients

The immunogenic and vaccine compositions of the invention can further comprise pharmaceutically acceptable carriers, excipients and/or stabilizers (see e.g. Remington: The Science and practice of Pharmacy (2005) Lippincott Williams), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as Mercury((o-carboxyphenyl)thio)ethyl sodium salt (THIOMERSAL), octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), TWEEN or PLURONICS.

The compositions of the invention can be in dosages suspended in any appropriate pharmaceutical vehicle or carrier in sufficient volume to carry the dosage. Generally, the final volume, including carriers, adjuvants, and the like, typically will be at least 1.0 ml. The upper limit is governed by the practicality of the amount to be administered, generally no more than about 0.5 ml to about 2.0 ml.

### Methods of Use

The invention encompasses methods of preventing and/or treating Hendra and/or Nipah virus infection comprising administering the immunogenic and vaccine compositions of the invention in any mammalian subject. Active immunity elicited by vaccination with a HeV and/or NiV G glycoprotein with the adjuvants described herein can prime or boost a cellular or humoral immune response. An effective amount of the HeV and/or NiV G glycoprotein or antigenic fragments thereof can be prepared in an admixture with an adjuvant to prepare a vaccine.

The invention encompasses methods of preventing and/or treating Hendra and/or Nipah virus infection in a human subject comprising administering an immunogenic and/or vaccine composition comprising a soluble HeV and/or NiV G glycoprotein or combinations thereof either by itself or in combination with at least one adjuvant suitable for use in humans. Adjuvants suitable for use in humans may be used alone or in combination. Examples of adjuvants suitable for use in humans include, but are not limited to, aluminum salts. Examples of aluminum salts include, but are not limited to, aluminum hydroxide, aluminium hydroxide gel (Alhydrogel™), aluminum phosphate, alum (potassium aluminum sulfate), or mixed aluminum salts. Additional examples of adjuvants suitable for use in humans include, but are not limited to, water-in-oil emulsions, oil-in-water emulsions, and AS04 (combination of aluminum hydroxide and monophosphoryl lipid A) and CpG oligodeoxynucleotides. CpG oligodeoxynucleotides are synthetic oligonucleotides that contain unmethylated CpG dinucleotides in particular sequence contexts (CpG motifs). These CpG motifs are present at a 20-fold greater frequency in bacterial DNA compared to mammalian DNA. CpG oligodeoxynucleotides are recognized by Toll-like receptor 9 (TLR9) leading to strong immunostimulatory effects. Suitable immunostimulatory oligonucleotides also include ODN (DNA-based) and ORN (RNA-based) oligonucleotides, which may have modified backbone including, without limitations, phosphorothioate modifications, halogenations, alkylation (e.g., ethyl- or methyl-modifications), and phosphodiester modifications.

The administration of a vaccine or immunogenic composition comprising HeV and/or NiV G glycoprotein with one or more adjuvants described herein, can be for either a prophylactic or therapeutic purpose. In one aspect of the present invention the composition is useful for prophylactic purposes. When provided prophylactically, the vaccine composition is provided in advance of any detection or symptom of HeV and/or NiV infection. The prophylactic administration of an effective amount of the compound(s) serves to prevent or attenuate any subsequent HeV and/or NiV infection.

When provided therapeutically, the vaccine is provided in an effective amount upon the detection of a symptom of actual infection. A composition is said to be “pharmacologically acceptable” if its administration can be tolerated by a recipient. Such a composition is said to be administered in a “therapeutically or prophylactically effective amount” if the amount administered is physiologically significant. A vaccine or immunogenic composition of the present invention is physiologically significant if its presence results in a detectable change in the physiology of a recipient patient, for example, by enhancing a broadly reactive humoral or cellular immune response to one or more strains of HeV and/or NiV. The protection provided need not be absolute (i.e., the HeV or NiV infection need not be totally prevented or eradicated), provided that there is a statistically significant improvement relative to a control population. Protection can be limited to mitigating the severity or rapidity of onset of symptoms of the disease.

A vaccine or immunogenic composition of the present invention can confer resistance to multiple strains of HeV and/or NiV. As used herein, a vaccine is said to prevent or attenuate an infection if its administration to a subject results either in the total or partial attenuation (i.e., suppression) of a symptom or condition of the infection, or in the total or partial immunity of the individual to the infection.

At least one vaccine or immunogenic composition of the present invention can be administered by any means that achieve the intended purpose, using a pharmaceutical composition as described herein. For example, administration of such a composition can be by various parenteral routes such as subcutaneous, intravenous, intradermal, intramuscular, intraperitoneal, intranasal, transdermal, or buccal routes. In one embodiment of the present invention, the composition is administered by subcutaneously. Parenteral administration can be by bolus injection or by gradual perfusion over time.

A typical regimen for preventing, suppressing, or treating a disease or condition which can be alleviated by a cellular immune response by active specific cellular immunotherapy, comprises administration of an effective amount of a vaccine composition as described above, administered as a single treatment, or repeated as enhancing or booster dosages, over a period up to and including one week to about twenty-four months. Non-limiting examples include a first dose followed by a second dose about at least 10, 11, 12, 13, 14, 15, 16, 17, 18, 19, 20, 21, 22, 23 or 24 days after the first dose (day 0). The amount of the dose of the immunogenic or vaccine composition may be the less than, the same as, or greater than the first dose administered at day 0.

According to the present invention, an “effective amount” of a vaccine or immunogenic composition is one which is sufficient to achieve a desired biological effect, in this case at least one of cellular or humoral immune response to one or more strains of HeV and/or NiV. It is understood that the effective dosage will be dependent upon the age, sex, health, and weight of the subject, kind of concurrent treatment, if any, frequency of treatment, and the nature of the effect desired. The ranges of effective doses provided below are not intended to limit the invention and represent examples of dose ranges which may be suitable for administering compositions of the present invention. However, the dosage may be tailored to the individual subject, as is understood and determinable by one of skill in the art, without undue experimentation.

The recipients of the vaccine and immunogenic compositions of the present invention can be any subject which can acquire specific immunity via a cellular or humoral immune response to HeV and/or NiV, where the cellular response is mediated by an MHC class i or class ii protein. Among mammals, the recipients may be mammals of the orders primata (including humans, chimpanzees, apes and monkeys). In one embodiment of the present invention there is provided a method of treating humans with the vaccine or immunogenic compositions of the invention. The subjects may be infected with HeV and/or NiV or provide a model of HeV or NiV infection as in experimental studies. In some embodiments, the subject is a domesticated mammal including, but not limited to, a horse, cow, oxen, water buffalo, sheep, pig (Mingyi (2010) Vet. Res. 41, 33), goat, dog (Biosecurity Alert—Hendra Virus Update, 27 Jul. 2011, Press Release, Biosecurity Queensland) or cat. In some embodiments, the subject is a fowl, including a chicken.

Vaccines of the present invention also provide for cross-protection against Nipah virus infection at doses used to protect against Hendra virus infection and thus also provide effective vaccination against Nipah virus.

Reference to an effective immune response should be understood as a reference to an immune response which either directly or indirectly leads to a beneficial prophylactic or therapeutic effect. In the case where the immunogen comprises a HeV or NiV G glycoprotein as described herein, such a response includes the reduction or blocking of viral reproduction and/or viral shedding and/or reduction in disease symptoms in an animal. It should be understood that efficacy is a functional measure and is not defined by reference to anti-HeV and/or anti-NiV antibody titre alone since the presence of circulating antibody alone is not necessarily indicative of the capacity of said circulating antibody to block viral reproduction and shedding.

Also by way of example, and not limitation, if a soluble G protein polypeptide of the invention is being administered to augment the immune response in a subject infected with or suspected of being infected with Hendra or Nipah and/or if antibodies of the present invention are being administered as a form of passive immunotherapy the composition can further comprise, for example, other therapeutic agents (e.g., anti-viral agents).

Example 4 below provides information on certain preferred compositions for use in vaccinating horses. In regard of other animals that may be infected with Hendra virus, and which therefore warrant vaccination to protect both animals and thus humans from both Hendra and Nipah virus infection, the following information is generally applicable and can readily be adapted by those skilled in the art. Generally speaking, companion animals (dogs and cats) warrant approximately 25 micrograms of Hendra antigen, and can benefit from an ISO adjuvant in the range of 25-150 micrograms, with a 5:1:1 ratio of saponin, phospholipid and sterol being among the preferred ISO compositions while using any of the component species as disclosed herein. For companion animals it is preferred that the final dose be about 1 ml. Polygen™ (MVP Technologies), a copolymer based adjuvant, may also be used at preferably about 5-15% (v/v).

Generally speaking, for larger farm animals (sheep, cows, pigs, etc.) the antigen and adjuvant dosing (and final dosing volume) amounts otherwise provided herein for horses are applicable, that is, from 50-250 micrograms of antigen, and typically about 250 micrograms of ISO may be used, final volume 1-3 ml for example). In regard of pigs, an alternative and effective adjuvant formulation involves (for approximately the same amount of antigen) a blend of ISO and ionic polysaccharide, specifically 100 mg DEAE dextran and 800 micrograms ISO in 1-3 ml final dose volume (again 5:1:1 of Quil A:phoshatidyl choline:cholesterol (see WO 2000/41720)).

### Differentiation of Vaccinated Animals

The invention also encompasses methods of differentiating healthy vaccinated animals from animals exposed to, or infected with HeV and/or NiV. During viral infection, HeV and NiV express additional proteins other than G glycoprotein (G) including fusion protein (F), matrix protein (M), phosphoprotein (P), large protein (L) and nucleocapsid protein (N). These additional proteins have the potential to induce immune responses in animals in the form of antibodies which bind to these proteins or T cell immunity. The level of antibody response to these other proteins can normally be measured by assays such as enzyme-linked immune assay (EIA). The immunogenic and vaccine formulations of the present invention, in some embodiments, contain only G glycoprotein as an HeV and/or NiV antigen and will therefore induce immune responses with antibodies only to the G glycoprotein of HeV and/or NiV. Animals vaccinated with the immunogenic compositions described herein which are subsequently infected by HeV or NiV will mount a booster immune response to the G glycoprotein, but will also show changes of antibody presentation to some other HeV and NiV proteins other than G glycoprotein. Thus, the presence of antibodies to any of the fusion protein (F), matrix protein (M), phosphoprotein (P), large protein (L) and nucleocapsid protein (N) can be measured in an EIA to determine the presence or absence of antibodies specific to these proteins in serum samples. If antibody to any of these other proteins (i.e. other than G glycoprotein) is detected, then the animal has been exposed to HeV and/or NiV. Alternatively, if no antibody to these other proteins is found and only antibodies binding to G protein are detected, then the animal has only been vaccinated.

The EIA of the present invention are both highly specific and highly selective in detecting and differentiating between animals infected with HeV and/or NiV and healthy animals which have been vaccinated with the immunogenic compositions described herein. The present invention may utilize a variety of assay procedures including ELISA in both homogenous and heterogenous environments. The assay procedures may be conducted on samples such as blood, serum, milk, or any other body fluid containing antibodies.

In some embodiments, the antibodies used in the EIA may uniquely compete with antibodies induced by vaccination with the G glycoprotein, but not antibodies induced in animals by infection with HeV and/or NiV. This allows not only serologic diagnosis of HeV and NiV infection, but differentiation of vaccination from infection in a single assay. The EIA procedure may be performed on standard blood serum samples or any body fluids or secretions containing antibodies. The EIA procedure may employ either monoclonal and/or polyclonal antibodies to G glycoprotein and any other HeV and/or NiV viral protein (e.g. fusion protein (F), matrix protein (M), phosphoprotein (P), large protein (L) and nucleocapsid protein (N) as such proteins are not present in vaccinated healthy animals which have not been exposed to HeV and/or NiV). The EIA may be carried out in any number of commercially available fixed or portable-manual, semi-automated or robotics-automated ELISA equipment with or without computer assisted data analysis reduction software and hardware. In some embodiments, the methods of differentiating healthy vaccinated animals from animals exposed to, or infected with HeV and/or NiV may be conducted on a biological sample isolated from a domesticated mammal including, but not limited to, a horse, cow, sheep, pig, goat, dog or cat. In some embodiments, the subject is a fowl, including a chicken. In some embodiments, the subject is a human.

### EXAMPLES

The following examples illustrate only certain and not all embodiments of the invention, and thus, should not be viewed as limiting the scope of the invention.

### Example 1

### Vector Constructs

Vectors were constructed to express transmembrane/cytoplasmic tail-deleted HeV G or NiV G. The cloned cDNA of full-length HeV or NiV G protein were amplified by PCR to generate fragments about 2600 nucleotides encoding the transmembrane domain/cytoplasmic tail-deleted HeV or NiV G protein.

The following oligonucleotide primers were synthesized for amplification of HeV G.

sHGS:
(SEQ ID NO: 5)
5′-GTCGACCACCATGCAAAATTACACCAGAACGACTGATAAT-3′.
sHGAS:
(SEQ ID NO: 6)
5′-GTTTAAACGTCGACCAATCAACTCTCTGAACATTGGGCAGGTAT
C-3′..

The following oligonucleotide primers were synthesized for amplification of NiV G.

sNGS:
(SEQ ID NO: 7)
5′-CTCGAGCACCATGCAAAATTACACAAGATCAACAGACAA-3′.
sNGAS:
(SEQ ID NO: 8)
5′-CTCGAGTAGCAGCCGGATCAAGCTTATGTACATTGCTCTGGTA
TC-3′..

All PCR reactions were done using Accupol DNA polymerase (PGS Scientifics Corp) with the following settings: 94° C. for 5 minutes initially and then 94° C. for 1 minute, 56° C. for 2 minutes, 72° C. for 4 minutes; 25 cycles. These primers generated a PCR product for the sHeV G ORF flanked by Sal 1 sites and the sNiV G ORF flanked by Xho 1 sites. PCR products were gel purified (Qiagen). After gel purification, sHeV G and sNiVG were subcloned into a TOPO vector (Invitrogen).

PSectag2B (Invitrogen) was purchased and modified to contain a S-peptide tag or a myc-epitope tag. Overlapping oligonucleotides were synthesized that encoded the sequence for the S-peptide and digested Kpn 1 and EcoR1 overhangs.

SPEPS:
(SEQ ID NO: 9)
5′-CAAGGAGACCGCTGCTGCTAAGTTCGAACGCCAGCACATGGATT
CT-3′.
SPEPAS:
(SEQ ID NO: 10)
5′AATTAGAATCCATGTGCTGGCGTTCGAACTTAGCAGCAGCGGTCT
CCTTGGTAC-3′..

Overlapping oligonucleotides were synthesized that encoded the sequence for the myc-epitope tag and digested Kpn 1 and EcoR1 overhangs.

MTS:
(SEQ ID NO: 11)
5′-CGAACAAAAGCTCATCTCAGAAGAGGATCTG-3′.
MTAS
(SEQ ID NO: 12)
5′-AATTCAGATCCTCTTCTGAGATGAGCTTTTGTTCGGTAC-3′.

64 pmol SPEPS and 64 pmol SPEPAS were mixed and heated to 65° C. for 5 minutes and cooled slowly to 50° C. 64 pmol MTS and 64 pmol MTAS were mixed and heated to 65° C. for 5 minutes and cooled slowly to 50° C. The two mixtures were diluted and cloned into Kpn1-EcoR1 digested pSecTag2B to generate S-peptide modified pSecTag2B or myc-epitope modified pSecTag2B. All constructs were initially screened by restriction digest and further verified by sequencing.

The TOPO sG construct was digested with Sal 1 gel purified (Qiagen) and subcloned in frame into the Xho 1 site of the S-peptide modified pSecTag2B or myc-epitope modified pSecTag2B. All constructs were initially screened by restriction digest and further verified by sequencing.

The Igκ leader-S-peptide-s HeVG (sG_{S-tag}) and the Igκ leader-myc tag-sHeVG (sG_{myc-tag}) constructs were then subcloned into the vaccinia shuttle vector pMCO2. Oligonucleotide SEQS: 5′-TCGACCCACCATGGAGACAGACACACTCCTGCTA-3′ (SEQ ID NO: 13) was synthesized and used in combination with oligonucleotide sHGAS to amplify by PCR the sG_{S-tag} and sG_{myc-tag}. All PCR reactions were done using Accupol DNA polymerase (PGS Scientifics Corp.) with the following settings: 94° C. for 5 minutes initially and then 94° C. for 1 minute, 56° C. for 2 minutes, 72° C. for 4 minutes; 25 cycles. These primers generated PCR products flanked by Sal 1 sites. PCR products were gel purified (Qiagen). After gel purification, sG_{S-tag} and sG_{myc-tag} were subcloned into a TOPO vector (Invitrogen). sG S-tag and sG myc-tag were digested with Sal 1 and subcloned into the Sal 1 site of pMCO2. All constructs were initially screened by restriction digest and further verified by sequencing. A codon optimized nucleotide sequence was subsequently generated to facilitate production in euckaryotic cell lines which is depicted in SEQ ID NO: 16.

For expression of the Hendra sG protein in CHO cells using the Chromos artificial chromosome expression (ACE) system, DNA encoding for the Hendra sG protein was amplified by PCR using Pfx polymerase (Invitrogen) according to manufacturer's instructions. The template was pCDNA Hendra sG (no S-peptide tag). The oligonucleotide primers used to amplify the DNA were: 5′-GATATCGCCACCATGGAAACCGACACCCTG-3′ (SEQ ID NO: 18) and 5′-GGTACCTCAGCTCTCGCTGCACTG-3′ (SEQ ID NO:19). Gel purification of the fragment was performed using QiaQuick gel extraction (Qiagen) following manufacturer's instructions. The PCR product was then ligated into Zero Blunt®TOPO® (Invitrogen), and the ligation mixture was transformed into One Shot Max efficiency cells (Invitrogen). DNA from a positive transformant was purified, and the sG insert was excised using Kpnl and EcoRV, and ligated into pCTV927, the ACE system targeting vector (ATV). Ligation reactions were then transformed into E. coli OmniMax cells (Invitrogen). Following identification of a positive clone, pCTV927/Hendra sG T1 plasmid was isolated, and the insert was then confirmed by sequencing.

### Example 2

### Protein Production of Soluble G Protein using CHO Cells

Chinese hamster ovary (CHO) ChK2 cells were thawed and transferred to a sterile 125 ml flask containing CD-CHO media (Invitrogen) and 6 mM Glutamax (Gibco), and subjected to passaging. One hour prior to transfection, the culture medium was removed and replaced with fresh ChK2 adherence culture medium. pCTV927/Hendra sG T1 plasmid was isolated, ethanol precipitated, and resuspended to a concentration of 0.85 μg/μL. The adherent cells were co-transfected with the ACE Integrase (pSI0343) and pCTV927/Hendra sG T1 with Lipofectamine™2000 (Invitrogen), according to manufacturer's instructions, using OptiMEM I (Gibco). The ACE Integrase consists of the integrase gene amplified from bacteriophage lambda DNA, but optimized for mammalian expression. The cultures were incubated overnight at 37° C./5% CO₂ with fresh ChK2 adherence media. The following day the culture media was removed, and cells were carefully washed with PBS, followed by the addition of 2 mL trypsin solution to detach the cells, and an additional 4 mL fresh ChK2 adherence media. The cells were then subjected to limiting serial dilution in 96-well plates followed by selection with 2 mg/mL Hygromycin, 24 hours after depositing in 96-well plates.

After 17 days of careful monitoring, 80 individual transfected clones were selected and dispensed into 24 well plates with 1 ml CD-CHO (Invitrogen) containing 6mM glutamax (Gibco) and 0.1 mg/ml hygromycin (maintenance selection media). Four days later the clones were split into new 24 plates as indicated below with maintenance selection media. Five hundred microliters (μl) of suspension culture were removed from each expression flask, and centrifuged at 500×g for 5 minutes. Supernatants were removed and transferred to clean fresh tubes, and frozen @-20° C. All supernates were later thawed and subjected to polyacrylamide gel electrophoresis (PAGE) using NuPAGE® Novex® Bis-Tris Mini Gels (Invitrogen). Two sets for each sample were run, one for gel staining and the other for Western blot analysis. The second set of gels was transferred onto nitrocellulose using an iBlot® gel transfer device (Invitrogen). An anti-G protein polyclonal antibody was used as the primary antibody, followed by a peroxidise-conjugated affinity purified anti-rabbit IgG antibody (Rockland). The blots were then developed by the addition of TMB Membrane Peroxidase substrate (KPL). Expression of the G protein was confirmed.

### Example 3

### Protein Production of Soluble G Protein using Vaccinia

For protein production the genetic constructs containing the codon optimized sequences were used to generate recombinant poxvirus vectors (vaccinia virus, strain WR). Recombinant poxvirus was then obtained using standard techniques employing tk-selection and GUS staining. Briefly, CV-1 cells were transfected with either pMCO2 sHeV G fusion or pMCO2 sNiV G fusion using a calcium phosphate transfection kit (Promega). These monolayers were then infected with Western Reserve (WR) wild-type strain of vaccinia virus at a multiplicity of infection (MOI) of 0.05 PFU/cell. After 2 days the cell pellets were collected as crude recombinant virus stocks. TK⁻ cells were infected with the recombinant crude stocks in the presence of 25 μg/ml 5-Bromo-2′-deoxyuridine (BrdU) (Calbiochem). After 2 hours the virus was replaced with an EMEM-10 overlay containing 1% low melting point (LMP) agarose (Life Technologies) and 25 μg/ml BrdU. After 2 days of incubation an additional EMEM-10 overlay containing 1% LMP agarose, 25 μg/ml BrdU, and 0.2 mg/ml 5-Bromo-4-chloro-3-indolyl-β-D-glucuronic acid (X-GLUC) (Clontech) was added. Within 24-48 hours blue plaques were evident, picked and subject to two more rounds of double selection plaque purification. The recombinant vaccinia viruses vKB16 (sHeV G fusion) and vKB22 (sNiV G fusion) were then amplified and purified by standard methods. Briefly, recombinant vaccinia viruses are purified by plaque purification, cell-culture amplification, sucrose cushion pelleting in an ultracentrifuge and titration by plaque assay. Expression of sHeV G was verified in cell lysates and culture supernatants.

### Example 4

### Protein Production of Soluble G Protein using 293F Cells

Genetic constructs containing the codon optimized sequences were used to transform 293F cells (Invitrogen) to produce a stable cell line which expresses HeV soluble G glycoprotein. CHO-S cells (Invitrogen) may also be used for transformation and expression of HeV soluble G glycoprotein. Transformed cells are plated on 162 cm² tissue culture flask with 35 ml DMEM-10. Cells were allowed to adhere and grow at 37° C. with 5-8% CO2 for several days. When cells were confluent, they were split into multiple flasks with DMEM-10 with 150 μg/ml Hygromycin B (30 ml per flask). When the cells are 70-80% confluent, they were washed twice with 30 ml PBS, then 20 ml of 293 SFM II (Invitrogen) was added and the cells were incubated at 37° C. with 5-8% CO₂ overnight. On the next day, cells were transferred into Erlenmeyer flasks with 200 ml SFM II media. Cells were allowed to grow at 37° C. with 5-8% CO₂ at 125 rpm for 5-6 days until cells started to die. At that time, the supernatant is collected.

Media from each Erlenmeyer flask is centrifuged at 3,500 rpm for 30 minutes. The supernatant was then transferred into 250 ml centrifuge bottles and spun at 10,000 rpm for one hour. The resulting supernatant is collected and protease inhibitor is added according to manufacturer's recommendation along with Triton X-100 to final concentration of 0.1%. The supernatant is then filtered through a 0.2 μm low protein binding filter membrane.

HeVsG is purified through use of an S-protein agarose affinity column. A 20 ml bed volume of S-protein agarose (Novagen) is loaded into a XK 26 column (GE Healthcare). The column is washed with 10× bed volumes of Bind/Wash buffer (0.15 M NaCl, 20 mM Tris-HCl, pH 7.5 and 0.1% Triton X-100). The prepared supernatant of HeV sG is applied to the column to maintain a flow rate of 3 ml/min. The column is washed with 10× bed volumes (200 ml) of Bind/Wash buffer I followed by 6× bed volumes (120 ml) of wash buffer 1× Wash Buffer (0.15 M NaCl, and 20 mM Tris-HCl, pH 7.5).

The pump is then stopped and the Wash Buffer is allowed to drain until it reaches the surface of the beads when 30 ml of Elution Buffer (0.2 M Citric Acid, pH 2) is added. The first 10 ml of flow through (this should still be the wash buffer) is collected and then the elution buffer is incubated with the beads for 10 minutes. Next, 15 ml of the eluate is collected into a 50 mL sterile conical centrifuge tube containing 25 ml of neutralization buffer (1 M Tris, pH 8). The pH is adjusted to neutral and the elution and incubation is repeated three times. All of the neutralized eluate is combined and concentrated to about 4 ml. The collected HeV sG (4 ml) is purified through a 0.2 μm low protein binding filter membrane (Acrodisc 13 mm Syringe Filter with 0.2 μm HT Tuffryn Membrane.

Gel Filtration can be utilized to further purify the HeV sG. After quality control analysis and confirmation of purity and oligomeric state, aliquot HeV sG pooled fractions of tetramer+dimer, dimer and monomer are stored at −80° C.

### Example 5

### Clinical Trial in Primates for Nipah Virus

Statistics. Conducting animal studies, in particular non-human primate studies, in biosafety level 4 (BSL-4) severely restricts the number of animal subjects, the volume of biological samples that can be obtained and the ability to repeat assays independently and thus limit statistical analysis. Consequently, data are presented as the mean or median calculated from replicate samples, not replicate assays, and error bars represent the standard deviation across replicates.

Viruses. NiV-Malaysia (GenBank Accession No. AF212302) was obtained from the Special Pathogens Branch of the Centers for Disease Control and Prevention, Atlanta, Georgia. NiV was propagated and titered on Vero cells as described for HeV in Rockx et al. (2010) J. Virol. 84, 9831.

Vaccine formulation. Three vaccine formulations of sGHeV were employed (10 μg, 50 μg or 100 μg). Production and purification of sGHeV was done as previously described in Pallister (2011) Vaccine 29, 5623. Each vaccine formulation also contained Alhydrogel™ (Accurate Chemical & Scientific Corporation) and CpG oligodeoxynucleotide (ODN) 2006 (Invivogen) containing a fully phosphorothioate backbone. Vaccine doses containing fixed amount of ODN 2006, varying amounts of sGHeV and aluminum ion (at a weight ratio of 1:25) were formulated as follows: 100 μg dose: 100 μg sGHeV, 2.5 mg aluminum ion and 150 μg of ODN 2006; 50 μg dose: 50 μg sGHeV, 1.25 mg aluminum ion and 150 μg of ODN 2006; and 10 μg dose: 5 μg sGHeV, 250 μg aluminum ion and 150 μg of ODN 2006. For all doses, Alhydrogel™ and sGHeV were mixed first before ODN 2006 was added. Each vaccine dose was adjusted to 1 ml with PBS and mixtures were incubated on a rotating wheel at room temperature for at least two to three hours prior to injection. Each subject received the same 1 ml dose for prime and boost and all vaccine doses were given via intramuscular injection.

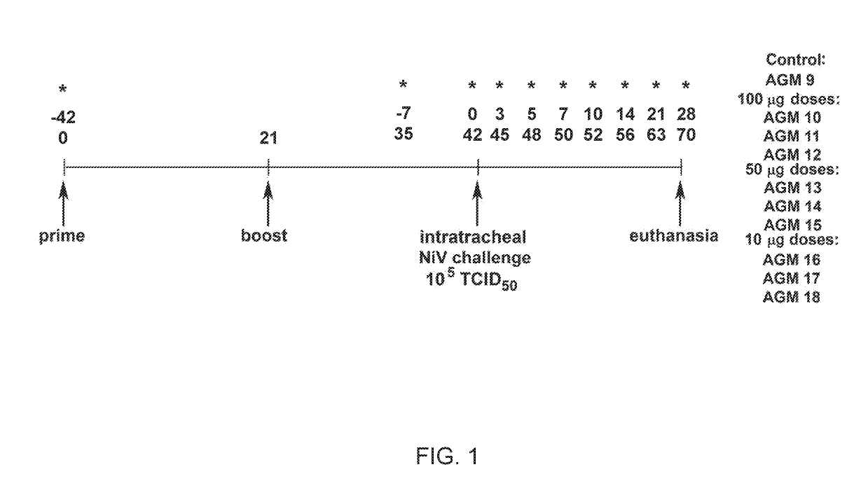

Animals. Ten young adult African Green Monkeys (AGM) (Chlorocebus aethiops), weighing 4-6 kg (Three Springs Scientific Inc.) were caged individually. Subjects were anesthetized by intramuscular injection of ketamine (10-15 mg/kg) and vaccinated with sGHeV on day −42 (prime) and day −21 (boost). Three subjects received two 10 μg doses (AGM 16, AGM 17, AGM 18), three subjects received two 50 μg doses (AGM 13, AGM 14, AGM 15), three animals received two 100 μg doses (AGM 10, AGM 11, AGM 12) and one subject (AGM 9) received adjuvant-alone. On day 0, subjects were anesthetized and inoculated intratracheally with 1×10⁵ TCID₅₀ (median tissue culture infectious dose) of NiV in 4 ml of Dulbecco's minimal essential medium (DMEM) (Sigma-Aldrich). Subjects were anesthetized for clinical examinations including temperature, respiration rate, chest radiographs, blood draw and swabs of nasal, oral and rectal mucosa on days 0, 3, 5, 7, 10, 14, 21 and 28 post-infection (p.i.). The control subject (AGM 9) had to be euthanized according to approved humane end points on day 10 post-infection. All other subjects survived until the end of the study and were euthanized on day 28 post-infection. Upon necropsy, various tissues were collected for virology and histopathology. Tissues sampled include: conjunctiva, tonsil, oro/naso pharynx, nasal mucosa, trachea, right bronchus, left bronchus, right lung upper lobe, right lung middle lobe, right lung lower lobe, light lung upper lobe, light lung middle lobe, light lung lower lobe, bronchial lymph node (LN), heart, liver, spleen, kidney, adrenal gland, pancreas, jejunum, colon transversum, brain (frontal), brain (cerebellum), brain stem, cervical spinal cord, pituitary gland, mandibular LN, salivary LN, inguinal LN, axillary LN, mesenteric LN, urinary bladder, testes or ovaries, femoral bone marrow. Vaccination was done under BSL-2 containment. A timeline of the vaccination schedule, challenge and biological specimen collection days is shown in FIG. 1.

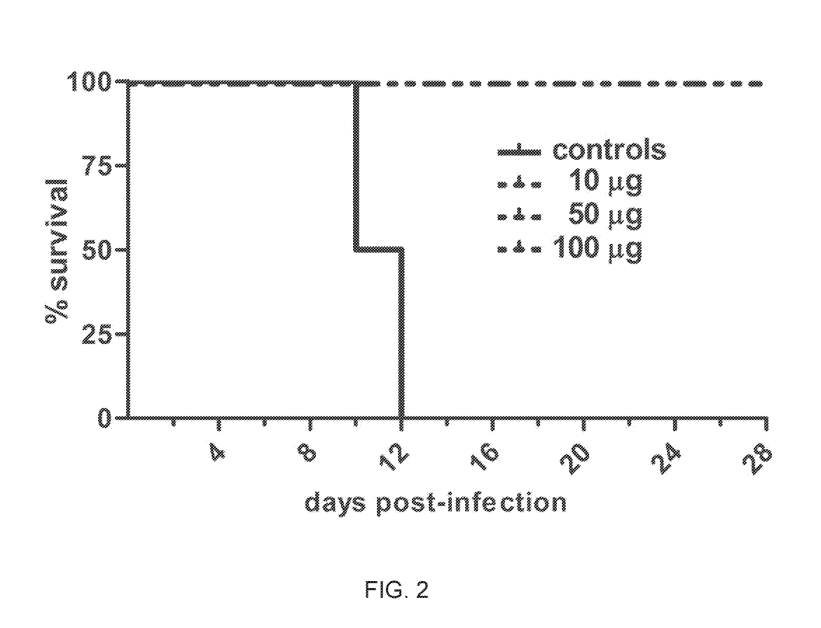

Vaccination and NiV challenge. Previously, we have demonstrated that intratracheal inoculation of AGMs with 10⁵ TCID₅₀ (median tissue culture infectious dose) of NiV caused a uniformly lethal outcome (Rockx et al. (2010) J. Virol. 84, 9831). Rapidly progressive clinical illness was noted in these studies; clinical signs included severe depression, respiratory disease leading to acute respiratory distress, severe neurological disease and severely reduced mobility; and time to reach approved humane endpoint criteria for euthanasia ranged from 7 to 12 days. Here we sought to determine if vaccination with sGHeV could prevent NiV infection and disease in AGMs. Doses of 10, 50 or 100 μg sGHeV were mixed with alum and CpG moieties as described in the Methods. Each vaccine formulation was administered subcutaneously to three subjects on day 0 (prime) and again on day 21 (boost) and one control subject (AGM 9) received an adjuvant alone prime and boost on the same days. On day 42, all subjects were inoculated intratracheally with 10⁵ TCID₅₀ NiV. The control subject (AGM 9) showed loss of appetite, severe sustained behavior changes (depression, decreased activity, hunched posture), decreases in platelet number and a gradual increase in respiratory rate at end-stage disease. Subsequently, AGM 9 developed acute respiratory distress and had to be euthanized according to approved humane end points on day 10 post-infection. In contrast, none of the vaccinated subjects had clinical disease and all survived until the end of the study. A Kaplan-Meier survival graph is shown in FIG. 2.

NiV-mediated disease in the control subject. Gross pathological changes in the control subject were consistent with those found previously in NiV-infected AGMs (Geisbert et al. (2010) PLoS One 5, e10690). Splenomegaly and congestion of blood vessels on surface of brain were present and all lung lobes were wet and heavy. NiV RNA and infectious virus were not recovered from AGM 9 blood samples and there was no evidence of viremia. AGM 9 had significant levels of NiV-specific IgM and detectable NiV-specific IgG and IgA. Further analysis of tissue samples revealed an extensive NiV tissue tropism similar to the wide-spread NiV infection seen previously in AGMs (Geisbert et al. (2010) PLoS One 5, e10690). AGM 9 had NiV RNA in the majority of tissues as indicated and infectious virus was recovered from numerous tissues. Significant lesions included interstitial pneumonia, subacute encephalitis and necrosis and hemorrhage of the splenic white pulp. Alveolar spaces were filled by edema fluid, fibrin, karyorrhectic and cellular debris, and alveolar macrophages. Multifocal encephalitis was characterized by expansion of Virchow-Robins space by moderate numbers of lymphocytes and fewer neutrophils. Smaller numbers of these inflammatory cells extended into the adjacent parenchyma. Numerous neurons were swollen and vacuolated (degeneration) or were fragmented with karyolysis (necrosis). Multifocal germinal centers of follicles in splenic white pulp were effaced by hemorrhage and fibrin, as well as small numbers of neutrophils and cellular and karyorrhectic debris. These findings were consistent with necrosis and loss of the germinal centers in the spleen. Extensive amounts of viral antigen were present in the brainstem highlight the extensive damage NiV causes in the central nervous system.

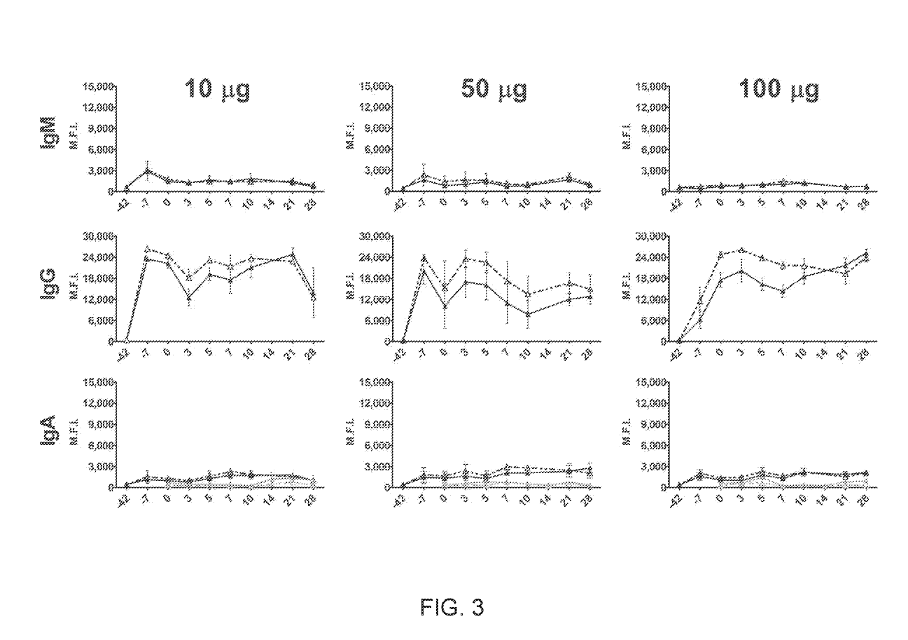

Protection of sGHeV-vaccinated subjects. All biological specimens, including all blood samples collected following challenge and all tissues collected upon necropsy, were negative for NiV RNA and infectious virus was not isolated from any specimen. Upon closer examination of tissue sections from vaccinated subjects, tissue architecture appeared normal and NiV antigen was not detected in any tissue using immunohistochemical techniques. To further dissect the vaccine-elicited mechanisms of protection, serum and mucosal sGNiV- and sGHeV-specific IgM, IgG and IgA as well as NiV and HeV serum neutralization titers were measured in vaccinated animals. As demonstrated in FIG. 3, seven days prior to challenge, subjects receiving the lowest sGHeV dose had detectable antigen-specific serum IgM and the highest level of sGHeV-specific serum IgG. Subjects given 50 μg sGHeV also had detectable levels of serum IgM and their highest levels of serum IgG seven days prior to challenge. High dose subjects had no detectable serum IgM and serum IgG levels were significantly less on day −7 as compared to the other two groups. By the day of NiV challenge, serum IgG levels in the high dose subjects had increased and all vaccinated subjects had similar IgG levels. Serum IgM levels did not change in any subject following NiV challenge. Serum IgG levels decreased in the medium dose subjects the day of NiV challenge and IgG levels decreased in low dose subjects just after NiV challenge. Interestingly, IgG levels increased in both of these groups by day 3 and day 5 p.i. but never surpassed the IgG levels present seven days prior to challenge and in both groups titer decreased significantly by day 28 p.i..

Conversely, serum IgG levels in the high dose group remained high and were at their highest of day 28 p.i.. Antigen-specific serum IgA was detectable in all subjects following vaccination; however, levels were very low and pre- and post-challenge levels did not appear to be significantly different (FIG. 3). A minimal increase in mucosal antigen-specific IgA was detected in nasal swabs from low dose subjects on day 14 p.i., however, the levels were so low these mucosal antibodies likely played no role in preventing the spread of NiV following challenge. Results from serum neutralization tests (SNTs) are shown in Table 1. For all vaccinated subjects, HeV-specific neutralization titer remained the same or decreased by day 28 p.i. and NiV-specific neutralization titer did not change significantly by day 7 p.i., even in subjects that had the lowest titer prior to challenge. One low dose and one high dose subject had a log increase in NiV SNT titer by day 14 p.i. and one medium dose subject had a log increase in NiV SNT titer by day 21 p.i. For all other vaccinated animals, changes in SNT titer were either inconsistent (titer would increase and then decrease) or insignificant (titer increased by 3-4 fold but not more than a log). Finally, seroconversion to the NiV fusion (F) envelope glycoprotein was measured in vaccinated subjects following NiV challenge. Minimal levels of serum anti-NiV F IgM were detected in the low and medium dose subjects on day 10 and day 21 p.i., respectively, and these low M.F.I. values suggest a weak primary antibody response following NiV challenge. Serum anti-NiV-F IgM was not detected in the high dose subjects suggesting these animals had little to no circulating virus following challenge.

TABLE 1

| | | Day² | −42 | −7 | 7 | 14 | 28 | −42 | −7 | 7 | 14 | 28 |
|---|---|---|---|---|---|---|---|---|---|---|---|---|
| sG_{HeV} doses | | AGM | HeV | | | | | NiV | | | | |
| 0 | μg³ | 9 | <20 | <20 | 24 | * | * | <20 | <20 | <20 | * | * |
| 10□ | μg | 16 | <20 | >2560 | >2560 | >2560 | 1074 | <20 | 379 | 226 | >2560 | 2147 |
| | | 17 | <20 | >2560 | >2560 | 905 | 537 | <20 | 134 | 134 | 537 | 453 |
| | | 18 | <20 | >2560 | >2560 | 453 | 537 | <20 | 189 | 134 | 189 | 453 |
| 50 | μg | 13 | <20 | >2560 | >2560 | >2560 | 757 | <20 | 379 | 189 | 189 | 453 |
| | | 14 | <20 | 1514 | >2560 | >2560 | 537 | <20 | 28 | 47 | 226 | 134 |
| | | 15 | <20 | 2147 | 757 | >2560 | 905 | <20 | 67 | 95 | 757 | 1074 |
| 100 | μg | 10 | <20 | >2560 | 2147 | 1810 | 453 | <20 | 67 | 113 | 268 | 453 |
| | | 11 | <20 | >2560 | >2560 | >2560 | 1514 | <20 | 134 | 189 | 905 | 1514 |
| | | 12 | <20 | >2560 | >2560 | >2560 | 757 | <20 | 189 | 226 | >2560 | 1514 |

### Example 6

### Clinical Trial in Swine for Nipah Virus Vaccine

A study will be conducted in pigs to evaluate the efficacy of an adjuvanted soluble Hendra virus G protein vaccine. The outline of the study is detailed in Table 2. The vaccine will consist of ˜100 ug/dose of soluble Hendra virus G protein, expressed and purified from CHO cells, according to the protocol of Example 2, and 10% SP-Oil adjuvant. For the purposes of this Example, the soluble G protein is provided as amino acids 73-604 of the native Hendra virus G glycoprotein (see SEQ ID NO: 2 and discussion thereof, see also SEQ ID NO: 2 in WO 2012/158643). Dimerization thereof occurs spontaneously, concomitant with expression from the cell line used. As expressed from CHO cells, resultant G protein fragment is approximately 50% dimer and 50% tetramer, with little remaining monomer. Expression in 293F cells leads to about 70% dimer. The 10% SP-Oil adjuvant components and final concentrations are: Pluronic L-121, 0.2%; squalane, 0.4%; and Tween-80®, 0.032%. Pigs will be vaccinated twice, by intramuscular administration (IM) at Day 0 and Day 21.

TABLE 2

| | Number | Vac- | Dates of | | Date of |
|---|---|---|---|---|---|
| Group | of Pigs | cine | Vaccination^{A} | Challenge | Challenge^{B} |
| T01 | 1 | PBS | Day 0; Day 21 | PBS | Day 35 |
| T02 | 1 | sG | Day 0; Day 21 | PBS | Day 35 |
| T03 | 2 | PBS | Day 0; Day 21 | 10⁵ PFU NiV | Day 35 |
| T04 | | sG | Day 0; Day 21 | 10⁵ PFU NiV | Day 35 |

^{A}Vaccine was administered intramuscularly (IM).
^{B}Challenge material was administered intranasally (IN).

The method of challenge will follow the published approach (Weingartl et al., 2006). The challenge virus will consist of plaque-purified NiV, re-isolated from the lungs of swine from previous inoculation experiments, and will be passed twice on Vero 76 cells. The piglets in this study will be challenged intranasally/orally with a total of 3 ml of 10⁵ PFU NiV.

The animals will be observed for any gross affects or abnormal clinical signs during daily feeding and cleaning activities. Rectal temperatures will be measured for first three days of acclimatization, for first three days post vaccination, and during sampling post challenge. Animal Records will be updated daily throughout the entire study. Blood samples from each animal will be collected at study days 0, 7, 14, 21, 28, 35, 36, 40, and prior to euthanasia on day 41 or 42. PBMC or serum will be separated from blood for further analysis (e.g. serum antibodies, immune cell profiles by flow cytometry focusing on CD4, CD8 and CD25 markers). Oral and nasal swabs will be collected prior to challenge at day −35, and post-challenge at day 1 and 4, and prior to euthanasia. The following samples will be collected in addition at post mortem: cerebrospinal fluid; trigeminal ganglion; olfactory bulb; cerebellum; forebrain; hind brain; turbinates; tonsil; trachea; lung lavage (BALF); lung; lung-associated lymph nodes; submandibular lymph nodes; mesenteric lymph nodes; small intestine; large intestine; kidneys; and urine, if available.

Serum samples for detection of neutralizing antibodies (by a microtiter plaque reduction neutralization assay) will be collected at study days 0, 7, 14, 21, 28, 35, 40, and prior to euthanasia on day 41 or 42. Blood for PBMC preparation will be collected on study days 0, 21, 28, 35, and at the time of euthanasia. CD4, CD8 and CD25 markers will be assessed by flow cytometry to determine changes in specific population frequencies and development of T-memory cells. Cells harvested on final bleed from the negative control pigs (vaccine, and farm control) will be used for additional in vitro experiments, looking at immune cells signalling due to NiV infection.

A recombinant soluble HeV G protein indirect ELISA will be used to determine antibody development in response to vaccination (along with an infected-cell lysate ELISA). The detection of selected cytokines (e.g. IFN-alpha, IFN-gamma, TNF-alpha) will be performed on bronchoalveolar lavage fluid (BALF). Viral RNA detection will be performed on all samples by real time RT-PCR, targeting the N gene. Virus isolation assays will be performed by plaque assay, and the presence of NiV will be confirmed either on supernatant from parallel wells, or by plaque immunostaining. Virus in formalin-fixed tissues will be detected by immunohistochemistry.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims.

1. An immunogenic composition comprising Hendra virus G glycoprotein, an adjuvant formulation, and one or more excipients, in an amount effective to elicit production of neutralizing antibodies against Nipah virus following administration to a subject, wherein said adjuvant formulation comprises a saponin, a sterol, a quaternary ammonium compound, a polyacrylic acid polymer, and a glycolipid.

2. The immunogenic composition of claim 1, wherein the adjuvant formulation further comprises an immunostimulatory oligonucleotide selected from DNA-based (ODN) and RNA-based (ORN) oligonucleotides.

3. The immunogenic composition of claim 2, wherein the final adjuvant formulation comprises: Quil A as saponin; cholesterol as sterol; dimethyl dioctadecyl ammonium bromide (DDA) as quarternary ammonium compound; CARBOPOL® as polyacrylic acid polymer; N-(2-Deoxy-2-L-leucylamino-b-D-glucopyranosyl)-N-octadecyldodecanoylamide hydroacetate as gylcoplipid; and CpG as immunostimulatory oligonucleotide.

4. The immunogenic composition of claim 1 wherein the soluble Hendra virus G glycoprotein consists of amino acids 73 to 604 of the native Hendra G glycoprotein (SEQ ID NO: 2).

5. The immunogenic composition of claim 4 wherein the soluble Hendra virus G glycoprotein is encoded by a nucleotide sequence comprising nucleotides 64 to 1662 of SEQ ID NO: 16.

6. The immunogenic composition of claim 1 wherein the soluble Hendra virus G glycoprotein is present in dimer form.

7. The immunogenic composition of claim 6 wherein each soluble Hendra virus G glycoprotein dimer subunit is connected by one or more disulfide bonds.

8. The immunogenic composition of claim 1 wherein the soluble Hendra virus G glycoprotein is present in tetramer form.

9. The immunogenic composition of claim 1 wherein the concentration of soluble Hendra virus G glycoprotein is about 5 to about 250 μg/ml.

10. The immunogenic composition of claim 1 wherein the subject is a human, horse, cow, sheep, pig, goat, chicken, dog or cat.

11. A method of producing a neutralizing antibody response against a Nipah virus in a subject comprising administering to the subject the immunogenic composition of claim 1 in an amount and duration effective to produce the neutralizing antibody response.

12. The method of claim 11 wherein the neutralizing antibody response reduces Nipah virus replication in the subject.

13. The method of claim 11 wherein the neutralizing antibody response reduces Nipah virus shedding in the subject.

14. The method of claim 11 wherein the subject has been exposed to Nipah virus.

15. The method of claim 14 wherein the subject is suffering from a Nipah virus infection.

16. The method of claim 11 wherein the immunogenic composition is administered by a route selected from the group consisting of intramuscular, intranasal and subcutaneous.

17. The method of claim 11 wherein the immunogenic composition is administered in a single dose, or is administered in multiple doses.

18. The method of claim 17 wherein the first dose is followed by a second dose at least about twenty-one days to about forty-two days after the first dose.

19. The method of claim 18 wherein each dose contains about 50 to about 250 μg of soluble Hendra virus G glycoprotein.

20. A method of differentiating a subject vaccinated with the immunogenic composition of claim 1 from a subject exposed to Nipah virus comprising detecting the presence of an antibody in a biological sample isolated from the subject against at least one of any of the following NiV viral proteins selected from the group consisting of fusion protein (F), matrix protein (M), phosphoprotein (P), large protein (L) and nucleocapsid protein (N).
